(19) United States
(12) Patent Application Publication
(10) Pub. No.: US 2026/0283632 A1
(43) Pub. Date: Sep. 24, 2026

(54) INTRAVASCULAR LITHOTRIPSY SYSTEM

(71) Applicant: Cardiovascular Systems, Inc., St. Paul, MN (US)

(72) Inventor: Joseph P. Higgins, Wayzata, MN (US)

(21) Appl. No.: 19/475,667

(22) PCT Filed: Apr. 19, 2024
(86) PCT No.: PCT/US2024/025362

§ 371 Date: Oct. 15, 2025

Related U.S. Application Data
(60) Provisional application No. 63/460,344, filed on Apr. 19, 2023.

(51) Int. Cl. A61B 17/22; A61B 90/00

(52) U.S. Cl. CPC A61B 17/22022; A61B 90/06; A61B 2017/22025; A61B 2017/22062; A61B 2090/064

(57) ABSTRACT

An intravascular lithotripsy system with an angioplasty-like balloon capable of being filled with a fluid, such as including saline that may be mixed or otherwise provided along with other fluid components like contrast as known. The system may include at least one emitter or pair of electrodes for generating a therapy. For example, an energy wave or high-pressure acoustic energy wave within the balloon with fluid inside may provide the therapy. The system also includes a sensor for detecting damage to the balloon, such as a rupture. The system further includes a control module with a processor for receiving information concerning balloon damage or rupture and the capability of preventing the system from energizing the emitters or electrodes once balloon damage or unacceptable degradation or imminent predicted failure is detected.

### CROSS-REFERENCE TO RELATED APPLICATIONS

This application claims priority to and the benefit of U.S. Provisional Patent Application No. 63/460,344, filed Apr. 19, 2023, the entire contents of which are incorporated herein by reference in their entireties.

### FIELD OF THE INVENTION

Catheters and systems for use in intravascular medical procedures for treating lesions such as calcified lesions.

### BACKGROUND

Calcification of blood vessels hinders proper blood flow and is associated with adverse health consequences including severe blood flow restriction. Calcification can range from minor to severe and calcification patterns can also vary significantly.

Intravascular lithotripsy is a medical procedure that uses shockwaves or high-energy acoustic waves to break up calcified plaque within a blood vessel. This procedure is commonly used to treat conditions such as peripheral artery disease, which can lead to poor circulation and increased risk of heart attack or stroke. Intravascular lithotripsy is also used in cardiac applications. However, traditional intravascular lithotripsy systems have some limitations, including the risk of damage to the balloon used in conjunction with shockwaves. In some cases, the balloon can rupture, leading to complications for the patient.

One successful approach to alleviating adverse effects of calcification is orbital atherectomy. While highly successful and considered the gold standard for treating difficult calcification challenges, other options are available to physicians for less challenging calcification patterns.

Intravascular lithotripsy (IVL) devices are available for some calcification patterns. Disposable IVL balloon devices are provided in different designs and sizes for peripheral or coronary indications. Commercially available designs utilize a reusable power source such as an IVL generator. One reusable generator comprises the following specification:

| | |
|---|---|
| Power | 110-240 VAC; 50-60Hz; Single Phase, 15A |
| | service |
| Size | 11″ (28.0 cm) high × 6″ (15.2 cm) wide × |
| | 11.5″ (29.2 cm) deep |
| Weight | 15 pounds (6.8 kg) |
| Output | Proprietary pulse delivery system. Output |
| | voltage 3000 volts peak, pulse |
| | frequency 1Hz |
| Mobility | Product is designed to be mounted to an IV |
| | pole |
| Length | 5 ft (1.53m) |
| Operation | Lithotripsy pulsing is activated by pushing a |
| | button on the Connector Cable. |
| Use | Re-usable |

The generator is used in conjunction with a disposable catheter. One such disposable device consists of a 0.014-inch guidewire-compatible, fluid-filled balloon angioplasty-like balloon catheter with two lithotripsy emitters incorporated into the shaft of the 12-mm-long balloon segment. A fluid filled angioplasty-like balloon (e.g. a 50/50 saline contrast medium) is inflated to about 4 atm and then electrical pulses are provided to the emitters that create high voltage sparks to provide the therapy. Acoustic waves are created and the calcium is fractured. Reported details of a known IVL balloon are illustrated in FIG. 6.

One example of an existing IVL therapy includes this treatment sequence:

| | |
|---|---|
| Treatment Frequency | 1 Pulse per Second |
| Maximum Number of Continuous Pulses | 20 Pulses |
| (1 cycle) | |
| Minimum Pause Time | 10 Seconds |
| Maximum Total Pulses Per Catheter | 160 (8 Cycles) |

Balloon life is a known problem associated with currently available IVL devices. Maximum pulse counts are associated with particular designs, with one device limiting the pulses to 300 pulses and another product limiting pulse counts to 160 per balloon. In the event the user attempts to deliver more than the maximum number of continuous pulses allowed, the IVL generator is designed to stop automatically. To resume pulsing, the user must wait at least a minimum pause time before resuming therapy. The therapy button must be released and pressed again to resume therapy. After a number of pulses far below the limits, deleterious material may be found within the saline filled balloon that is caused by the electrical pulses and generated energy (bubble creation, collapse, heat, shockwaves).

If a balloon ruptures or otherwise fails during therapy pulsing, IVL devices of currently available systems are at the mercy of a human operator to identify balloon failure and stop the therapy. If a current system experiences failure at the beginning of a pulse cycle, unless a human identifies the failure and intervenes, the system will continue to provide a therapy session.

An example of a balloon integrity monitoring system is disclosed in US publication no. 20210378743, published on Dec. 9, 2021. However, this system is complex and includes a crossing profile increasing design that can be detrimental to the crossing profile of the system, as well as the system stiffness and cost.

### SUMMARY

The present invention is an intravascular lithotripsy system with an angioplasty-like balloon capable of being filled with a fluid, such as saline. It is known to provide and use saline as an inflation fluid, as such can be a component of the fluid. Other fluids like contrast are also known to be mixed or provided with the saline. The system may include at least one emitter or pair of electrodes for generating a therapy. For example, an energy wave or high-pressure acoustic energy wave within the balloon with fluid inside may provide the therapy. The system also includes a sensor for detecting damage to the balloon, such as a rupture. The system further includes a control module with a processor for receiving information concerning balloon damage or rupture and the capability of preventing the system from energizing the emitters or electrodes once balloon damage or unacceptable degradation or imminent predicted failure is detected.

In another embodiment, the intravascular lithotripsy system described herein includes an angioplasty-like balloon capable of being filled with a fluid, at least an emitter or pair of electrodes for generating an energy wave or high-pressure acoustic energy wave within the balloon with fluid inside, a sensor, located in a proximal region of the catheter assembly, for detecting damage to the balloon or other failure or imminent failure, and a control module with a microprocessor for receiving information concerning balloon damage or rupture or failure and preventing the system from energizing the emitters or electrodes. This system can optionally assist physicians in avoiding undesirable balloon rupture by automatically communicating possible imminent failure of the balloon.

In one aspect of the present invention, the intravascular lithotripsy (IVL) system comprises a catheter having a distal end provided with an inflatable balloon and at least one emitter for creating energy waves within the balloon for conducting and IVL operation and to provide therapy to a lesion of a subject's vasculature, a sensor that is operatively associated with the balloon for monitoring a condition of the balloon and for detecting an undesirable level of damage to the balloon, and a control system operatively connected with the sensor and the emitter for preventing the emitter from creating further energy waves and further IVL operations when the sensor determines an undesirable level of damage to the balloon.

The sensor can comprise a pressure sensor that monitors fluid pressure within one of the balloon or a pressure fluid lumen of the catheter that provides inflation fluid to the balloon to either directly or indirectly monitor the fluid pressure within the balloon. The pressure sensor can be provided to a catheter shaft portion of the IVL system within the balloon for directly monitoring fluid pressure within the balloon. Alternatively, the pressure sensor can be provided at a component of the catheter that is to be positioned outside of the subject's vasculature and is connected indirectly with the balloon by way of the pressure fluid lumen of the catheter for inflation of the balloon.

The system can further comprise a high voltage pulse generator electrically connected with the emitter and for creating a spark within the balloon at the emitter when a high voltage pulse is generated by a therapy initiation control module.

The system can further comprise an indicator feature that provides information to a user of at least the undesirable damage to the balloon, when detected.

The system can comprise a plurality of sensors for monitoring the balloon and for determining the undesirable level of damage to the balloon. A control module can also be provided that includes a processer and memory, the memory including programming for controlling a fault detection algorithm.

At least one sensor can alternatively comprise an optical sensor that senses deleterious material within the inflation fluid, the presence of such deleterious material being correlated with imminent balloon failure. At least one sensor can comprise a MEMS pressure sensor.

In another aspect of the present invention, a method of using an intravascular (IVL) system having a firing prevention feature can comprise inserting an angioplasty-like balloon within a vessel of a subject's vasculature in the region of a lesion; at least partially expanding the balloon; firing the IVL system to produce energy waves within the angioplasty-like balloon to provide therapy to the lesion; monitoring a condition of the angioplasty balloon; detecting an undesirable level of damage to the balloon; and thereafter preventing firing of the system after the undesirable level of damage to the balloon is detected.

The method can further include a step of providing indicia to a user indicating that the IVL system is preventing further therapy using the IVL system and the creation of energy waves within the balloon.

The various inventions disclosed herein address these, inter alia, issues.

### BRIEF DESCRIPTION OF THE SEVERAL VIEWS OF THE DRAWINGS

These drawings are exemplary illustrations of certain embodiments and, as such, are not intended to limit the disclosure.

FIG. 1 schematically illustrates a system including a sensor according to the present invention.

FIG. 2 schematically illustrates an inflated balloon with a sensor in a vessel for providing intravascular lithotripsy according to an aspect the present invention.

FIG. 3 is a schematic flowchart showing a method according to another aspect of the present invention.

FIG. 4 is a schematic cross section of a catheter connector according to an embodiment of the present invention.

FIG. 5 is a schematic illustration of another embodiment of the present invention showing a system with a plurality of wave generating electrode pairs.

FIG. 6 is an illustration of features of certain IVL catheters showing details of a working length of such an IVL catheter, an energy profile of the IVL catheter, and the provision of an optimized overlap zone within such an IVL catheter design.

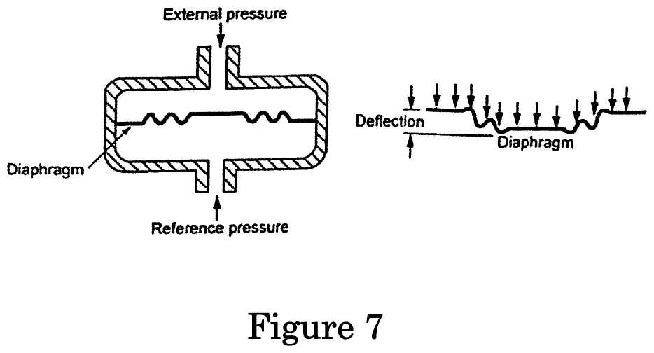

### DETAILED DESCRIPTION

Unless the context appears otherwise, the following terms or phrases shall have the following meaning (in both this singular and plural):

The terms “conductor”, “conductive” and “conducting” mean electrically conductive, and refer to materials that easily permit the flow of electrical current through such material. Conductive materials may, in some examples, be thermally conductive, but are not always so. Materials such as carbon black, gold, and metals are representative conductive materials.

The phrase “conductive path” refers to a viable path for the flow of electrical current along such path and may, for example, refer to an entire surface of a balloon on component thereof coated with a conductive material, or only a portion of a surface coated with a conductive material such as conductive stripes or predetermined (e.g. rectangular) shapes on the surface or layer of a balloon. Examples of ink printed medical devices are described in U.S. Pat. Nos. 5,836,874; 7,379,767; 9,7636,24; 9,913,594 and 10,751,000.

The phrase “intravascular lithotripsy device” includes devices that create acoustic waves by spark or arcing discharges between electrode components but may also include devices that create acoustic energy within the balloon via laser energy sources. Examples of such laser systems are described in U.S. Pat. Nos. 11,058,492 and 11,246,569 (the entire contents of which are incorporated by reference). Examples of electrically induced systems are described in U.S. Pat. Nos. 8,728,091, 9,642,673 and 10,850,078 and Published U.S. Pat. Appl. No. 2022-0054194 (the entire contents of which are incorporated by reference). Any suitable source of energy may be used to create the shockwave or acoustic wave. Examples include laser-based systems, spark generating systems, ultrasonic systems, high intensity focused ultrasound (HIFU) systems and the like.

The phrase “lithotripsy emitters” means electrode-based systems and may also include laser or optical systems.

The phrase “angioplasty balloon material” means any material previously used for an angioplasty balloon, including compliant, semi-compliant and non-compliant balloons. Such materials include materials such as a composite or multilayer structure. Compliant materials include silicone, polyurethane or nitinol materials. In some embodiments, angioplasty balloon materials may comprise balloon in balloon designs and the fluids filling the inner balloon may be dissimilar to the fluid filling the outer balloon to exploit differences in insulative properties, and speeds of sound of materials. Compliant, semi-compliant, and non-compliant materials may include nylon, polyurethanes, silicones, polyethylene terephthalate (PET) and other biocompatible materials. Recommended burst pressure of angioplasty balloons is typically between 4 and 20 atm, more preferably between 8 and 12 and one example is a Nylon 12 material with a rated burst pressure of between about 9 and 12 atm.

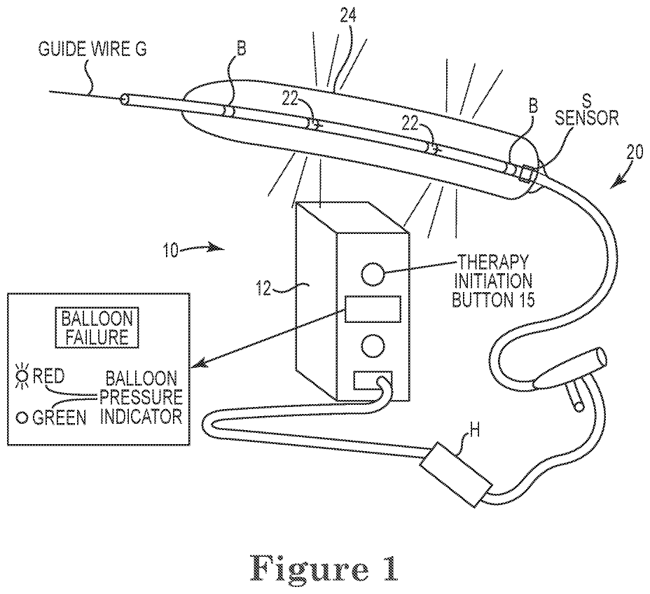

With reference to the Figures, FIG. 1 shows a system 10 according to the present invention comprising a power source 12 (in the form of an electrical generator, but alternatively in the form of a laser or ultrasound system), an optional handle H, a therapy delivery control 15 and an over the wire G catheter 20 (e.g. disposable) with two lithotripsy emitters 22 (shown in the form of arcing electrodes, but alternatively they could comprise optical or laser emitters or ultrasonic emitters), and a fluid fillable balloon 24. The housing of the power source 12 may also house a control module with a microprocessor as described in greater detail below. Optional imaging marker bands B may be provided. The system also includes a sensor S for detecting balloon 24 failure. The sensor in this embodiment is shown as mounted on the shaft of a catheter member that includes a passage or lumen for a guidewire G.

The emitters or electrodes 22 are capable of generating shockwaves or high-energy acoustic waves within the balloon 24. The emitters or electrodes can be positioned within the balloon, along a shaft member for holding the energy delivery components. They may be linearly placed along the longitudinal axis of the system, or at different radial angles to provide more of a 360 degree effective treatment. In another embodiment, a shockwave can be generated outside of the body and the balloon may include an energy deflection or focusing element for receiving the energy and directing it toward the lesion.

The balloon 24 may be placed in a deflated position so as to more readily pass through a patient's vasculature to arrive at the scene of calcification. In use within the body, the balloon 24 is advanced to the treatment site and then will be inflated to a common pressure for angioplasty procedures (e.g. 4 atm) and the therapy actuated via a user activating (e.g. pressing a button) the delivery control 15. Often there will be several (e.g. 8 or more) therapy sessions prior to exceeding the maximum number of therapy sessions allowed.

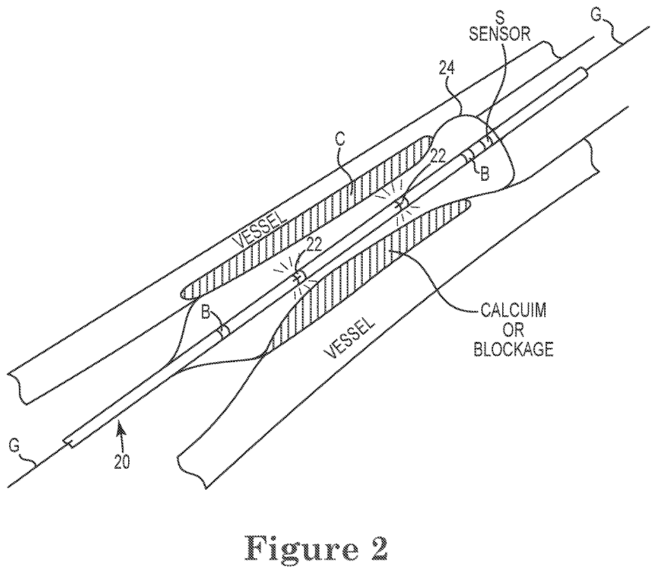

FIG. 2 shows the balloon 24 inflated to a therapy delivery state where the lithotripsy emitters 24 may be “fired” to disrupt the calcium C. Optional indicator bands B may be provided to afford visualization and proper positioning by use of known imaging techniques. The balloon 24 is inflated to a typical angioplasty pressure (e.g. 4 atm) and therapy is delivered. The balloon 24 may naturally expand due to its internal pressure during or just after the therapy is delivered (as the calcium block is broken up or fractured) to clear the vessel for passage of blood.

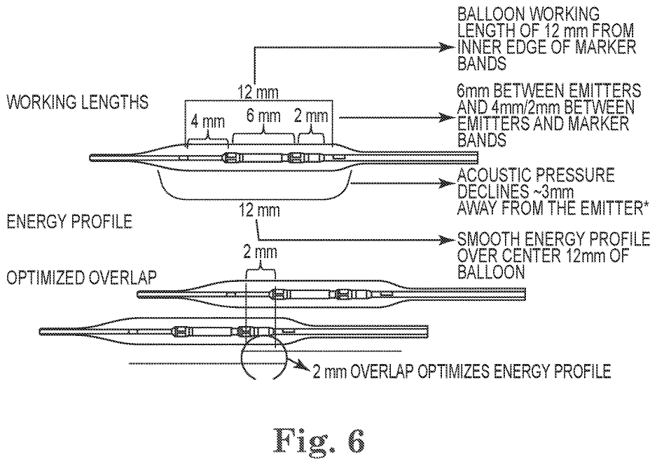

The material of the balloon 24 is inflated well below its burst pressure (e.g. 10 atm) to avoid balloon failure. The system also includes the sensor S for detecting damage to the balloon, such as a rupture. The location of the sensor S may be different in different embodiments. It could be located on the catheter shaft as shown in FIG. 1, or it could be on the balloon material itself. For example, a conductive path to a balloon mounted sensor may be provided by ink printed circuit components. Alternatively, the sensor S could be located near a proximal portion of the disposable catheter 20 as discussed below in conjunction with FIG. 5.

The sensor S should be located in a position where it will not be subject to undue interference from a therapy energy regimen. Shields or baffles may be employed to protect some pressure sensors located in the balloon 24 from therapy energy that might otherwise damage the sensor.

The sensor S can be a pressure sensor that detects sudden pressure drops or a reduction in balloon pressure below a predetermined level. Other types of sensors, such as strain gauges or temperature sensors, can also be used.

The pressure sensor may optionally be correlated to measure the pressure of the expansion of flexible tubing to the pressure sensor. Mounting of the pressure sensor on a compliant tube section and/or compliant diaphragm mounted on the tubing section, vs directly in the fluid affords indirect vs. direct measurement of the viscous (contrast/saline mix) inside the lumen. This tubing expansion vs. the pressure sensor will allow for no direct fluid transfer and/or contact between the sensor S and the fluid medium in the sensor S, nor the electrical signals within an electrically coupled balloon. This type of measurement is seen in diaphragm sensors:

Some contrast medium is similar to sugar water and can contaminate the pressure sensors. This tubing expansion vs. the pressure sensor will allow for no direct fluid transfer and/or contact between the sensor S and the fluid medium in the sensor, nor the electrical signals within an electrically coupled balloon. The pressure signals in the lumen may be monitored for unacceptable or ominous decay rates indicating loss of bond or holes/rupture of the balloon.

The pressure in the balloon may also be monitored to gage fluid volume in the system. As the balloon expands through activation of the IVL system the volume in the system will increase with a reduction in pressure in the system. The continued decay/rupture vs. pressure increase/decrease via balloon volume changes may be differentiated via pressure profile changes.

A pressure sensor may detect a failure in an angioplasty-like balloon system, for example, by:

Overinflation: One of the most common failures in angioplasty balloons is overinflation, which can cause the balloon to burst. A pressure sensor placed inside the balloon can directly detect the pressure inside the balloon and alert the medical team if the pressure exceeds a predetermined level. This can help prevent the balloon from bursting and causing injury to the patient.

Leakage: Another potential failure mode for angioplasty balloons is leakage, which can occur if there is a defect in the balloon material or if the balloon is not properly inflated. A pressure sensor can detect a rate of pressure drop or drop in pressure inside the balloon to a threshold, indicating that there is a leak, and alert the medical team. This can help ensure that the balloon is replaced before the procedure continues, reducing the risk of complications.

Overall, pressure sensors can play a role in ensuring the safety and efficacy of lithotripsy procedures by detecting potential failures in the balloon and alerting the medical team to take appropriate action.

The sensor S is preferably connected to a microprocessor that receives information about balloon damage or rupture. The system 10 can include a control module that may include a hardware microprocessor and memory as operatively connected together, the memory including programming that may comprise software or firmware for controlling any number of fault detection algorithms or tests when executed by the processor such as those described herein. The processor P can be programmed to prevent the system 10 from energizing the emitters or electrodes if there is a risk of further damage to the balloon, balloon failure or imminent balloon failure. For example, if the sensor S detects a sudden pressure drop, the microprocessor can automatically stop the delivery of shockwaves or high-energy acoustic waves to prevent further damage to the balloon and any attendant unwanted consequences for the patient. In addition to the fast drop, the pressure in the system (catheter and balloon) can be monitored for high pressure spikes indicating a kink or occlusion, or a slow leak preemptively indicating the balloon may be suspect and decreasing pressure. The processor may be partially or completely located in the console of the power source or alternatively, the handle H, or alternatively portions may be placed in the catheter itself for possible response time advantages.

Optionally, the microprocessor may be programmed to temporarily shut off the pressure monitoring system during the very short duration of a therapy energy pulse. This tactic may be employed to help filter interference from the system.

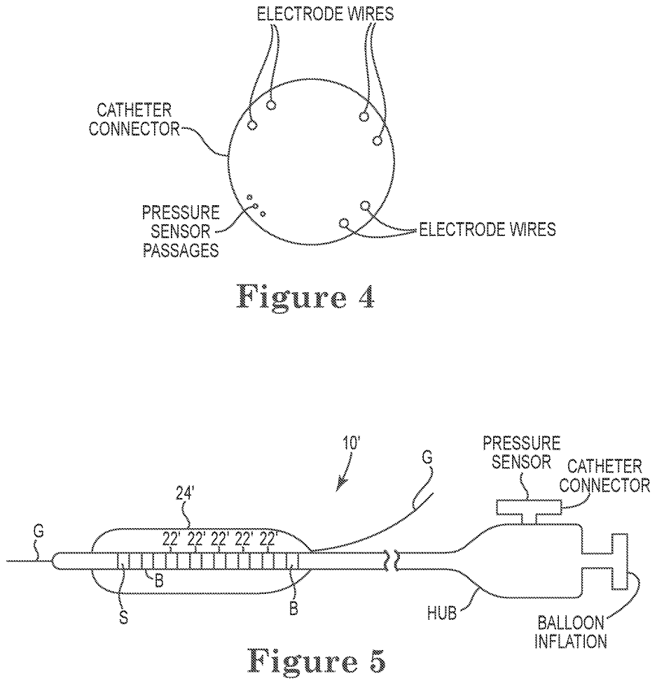

The location of the sensor S may vary. Referring to FIG. 5, a system 10′ is shown having a plurality (5) of emitters 22′, a slightly different guidewire G system, balloon 24′ and a sensor S near a proximal DISTAL portion of the catheter. FIG. 4 shows a cross section of a catheter connector that illustrates one embodiment of the location of wires for the emitters and passageways for the pressure sensor and fluid for the balloon. Such wires comprise a portion of the conductive path for the delivery of energy to the emitters.

FIG. 5 also shows an optional or alternative sensor S″ in the proximal portion of the catheter assembly in the form of a pressure sensor.

In one embodiment, the pressure sensor is in the hub (outside the body) of the catheter assembly 20′. Given the balloon may be semi rigid/rigid and the fluid is incompressible (minus air) the pressure outside the body will be the same as the pressure inside the body. This location has the advantage that it allows for the pressure sensor to be reuseable. This will also allow for the sensor S to be relatively free of interference from electrical signals and high energy pulses that may occur during therapy activation.

The sensor(s) S may be comprised of MEMS or optical membrane fiber such as a handle pressure sensor for PVAD pressure lumen monitoring. This embodiment of pressure sensing element S would be running in series with the pressure inflation lumens of the device in which the off the shelf indeflator. Inflation and adjustment of the inflation of the balloon may still be controlled and managed with that off the shelf endeflator.

In another embodiment, the sensor S could comprise an optical sensor for detecting the presence of eroded material from electrodes and other deleterious matter whose presence can be correlated to imminent balloon failure.

It should be noted that the processor or microprocessor may be programmed to identify balloon failure or imminent balloon failure by processing information from a variety of sources. For example, the system may count the number of therapy pulses provided for a particular balloon; it may also have an optical detector for detecting the presence of deleterious material within the balloon from electrode wear and finally, and the system's processor may also receive information from a pressure sensor S. If the device approaches (but does not exceed) the maximum number of pulses per balloon but also detects deleterious matter within the balloon and a drop in balloon pressure, then the system may automatically shut off despite being below the therapy limit as an additional safeguard beyond the simple pulse count.

Commercially available IVL devices operate at a voltage of slightly less than 3000 volts and include current of between 20 and 300 amps. The present invention may afford the use of higher voltages and/or currents or power while safely protecting the patient. The control module may afford higher energy therapy signals and adjust the useful life of the balloon downwardly accordingly. The present invention also reduces the strain on the physician operator by providing less concerns to distract a physician during a procedure.

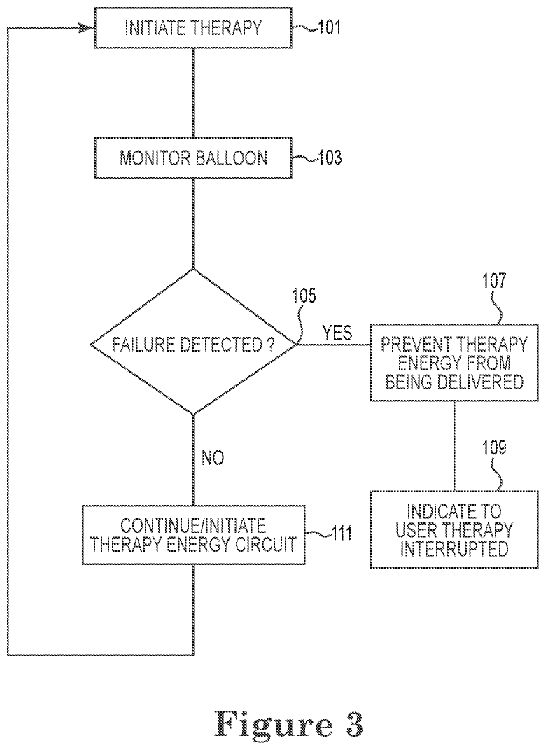

Referring now to FIG. 3, in another aspect of the present invention, the invention includes a method of using an intravascular system having a therapy delivery prevention feature comprising: inserting an angioplasty-like balloon within a vessel in the region of a lesion; at least partially expanding the balloon 24 (e.g. see FIG. 2); providing therapy in step 101 to the system to produce shockwaves or high energy acoustic waves within the angioplasty-like balloon to provide therapy to the lesion; monitoring the state of the angioplasty-like balloon 103; detecting an undesirable level of damage to the balloon in step 105; and preventing firing of the system in step 107 after the undesirable level of damage to the balloon is detected. Optionally, the system may inform a user in step 109 about the interruption in therapy energy availability. If no balloon failure is detected the system 10 can continue to enable therapy energy delivery in step 111.

The intravascular lithotripsy system and methods described herein can help physicians avoid undesirable balloon rupture by automatically communicating possible imminent failure of the balloon. This feature can provide an added level of safety for patients undergoing intravascular lithotripsy procedures.

The devices illustrated in the drawings illustrate exemplary systems for use of the present invention. It is noted that the present invention may also be used with the systems described in the following patent applications (the entire contents of which are incorporated by reference):
1. U.S. Provisional Patent Application No. 63/434,912; INTRAVASCULAR LITHOTRIPSY DEVICES AND SYSTEMS HAVING SPARK MONITORING FEEDBACK; Filing Date: Dec. 22, 2022; or
2. U.S. Provisional Patent Application No. 63/425,169; INTRAVASCULAR LITHOTRIPSY DEVICES AND SYSTEM, Filing Date: Nov. 14, 2022
3. U.S. Provisional Patent Application No. 63/416,231; CATHETER SYSTEM WITH FORWARD FACING ELECTRODES FOR CREATING ENERGY WAVES; Filing Date: Oct. 14, 2022.
4. U.S. Provisional Patent Application No. 63/458,728; INTRAVASCULAR LITHOTRIPSY DEVICES AND SYSTEMS WITH ENERGY WAVE DETECTION; Filing Date: Apr. 12, 2023.

The present invention is also particularly suitable for use in a forward firing (e.g. axial) system as described in the above patent applications.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Thus, instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “microprocessor” or “processor,” as used herein may refer to any of the foregoing structure or any other structure suitable for implementation of the techniques described herein. Also, the techniques could be fully implemented in one or more circuits or logic elements.

Thus, a medical device or system has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. An intravascular lithotripsy (IVL) system comprising a catheter having a distal end provided with an inflatable balloon and at least one emitter for creating energy waves within the balloon for conducting an IVL operation and to provide therapy to a lesion of a subject's vasculature, a sensor that is operatively associated with the balloon for monitoring a condition of the balloon and for detecting an undesirable level of damage to the balloon, and a control system operatively connected with the sensor and the emitter for preventing the emitter from creating further energy waves and further IVL operations when the sensor determines an undesirable level of damage to the balloon.

2. The system of claim 1, wherein the sensor comprises a pressure sensor that monitors fluid pressure within one of the balloon, or a pressure fluid lumen of the catheter that provides inflation fluid to the balloon to either directly or indirectly monitor the fluid pressure within the balloon.

3. The system of claim 2, wherein the pressure sensor is provided to a catheter shaft portion of the IVL system within the balloon for directly monitoring fluid pressure within the balloon.

4. The system of claim 2, wherein the pressure sensor is provided at a component of the catheter that is to be positioned outside of the subject's vasculature and is connected indirectly with the balloon by way of the pressure fluid lumen of the catheter for inflation of the balloon.

5. The system of claim 1, further comprising a high voltage pulse generator electrically connected with the emitter and for creating a spark within the balloon at the emitter when a high voltage pulse is generated by a therapy initiation control module.

6. The system of claim 1, further comprising an indicator feature that provides information to a user of at least the undesirable damage to the balloon, when detected.

7. The system of claim 1, comprising a plurality of sensors for monitoring the balloon and for determining the undesirable level of damage to the balloon.

8. The system claim 1, wherein the control module includes a processer and memory, the memory including programming for controlling a fault detection algorithm.

9. The system of claim 1, wherein the sensor comprises an optical sensor that senses deleterious material within the inflation fluid, the presence of such deleterious material being correlated with imminent balloon failure.

10. The system of claim 1, wherein the sensor comprises a MEMS pressure sensor.

11. A method of using an intravascular (IVL) system having a firing prevention feature comprising: inserting an angioplasty-like balloon within a vessel of a subject's vasculature in the region of a lesion; at least partially expanding the balloon; firing the IVL system to produce energy waves within the angioplasty-like balloon to provide therapy to the lesion; monitoring a condition of the angioplasty balloon; detecting an undesirable level of damage to the balloon; and thereafter preventing firing of the system after the undesirable level of damage to the balloon is detected.

12. The method of claim 11 further including providing indicia to a user indicating that the IVL system is preventing further therapy using the IVL system and the creation of energy waves within the balloon.

13. The method of claim 11, wherein the sensor comprises a pressure sensor that monitors fluid pressure within one of the balloon, or a pressure fluid lumen of the catheter that provides inflation fluid to the balloon to either directly or indirectly monitor the fluid pressure within the balloon.

14. The method of any of claim 13, wherein the pressure sensor is provided to a catheter shaft portion of the IVL system within the balloon for directly monitoring fluid pressure within the balloon.

15. The method of claim 13, wherein the pressure sensor is provided at a component of the catheter that is to be positioned outside of the subject's vasculature and is connected indirectly with the balloon by way of the pressure fluid lumen of the catheter for inflation of the balloon.

16. The method of claim 11, further comprising a high voltage pulse generator electrically connected with the emitter and for creating a spark within the balloon at the emitter when a high voltage pulse is generated by a therapy initiation control module.

17. The method of claim 11, comprising a plurality of sensors for monitoring the balloon and for determining the undesirable level of damage to the balloon.

18. The method of claim 11, wherein the control module includes a processer and memory, the memory including programming for controlling a fault detection algorithm.

19. The method of claim 11, wherein the sensor comprises an optical sensor that senses deleterious material within the inflation fluid, the presence of such deleterious material being correlated with imminent balloon failure.

20. The method of claim 11, wherein the sensor comprises a MEMS pressure sensor.
